(19) United States
(12) Patent Application Publication
(10) Pub. No.: US 2008/0094575 A1
(43) Pub. Date: Apr. 24, 2008

(54) Apparatus and Methods for Demonstrating the Effects of Anti-Reflective Lens Coatings

(76) Inventor: Brian W. Crain, Clarksville, TN (US)

Correspondence Address: PATENT DEPARTMENT;LARKIN, HOFFMAN, DALY & LINDGREN, LTD., 1500 WELLS FARGO PLAZA, 7900 XERXES AVENUE SOUTH, BLOOMINGTON, MN, 55431, US

(21) Appl. No.: 11/575,628

(22) PCT Filed: Sep. 21, 2005
(86) PCT No.: PCT/US05/33866

§ 371 Date: Mar. 20, 2007

Related U.S. Application Data
(60) Provisional application No. 60/611,638, filed on Sep. 21, 2004.

(51) Int. Cl. A61B 3/04

(52) U.S. Cl. 351/229; 351/246

(57) ABSTRACT

Apparatus and methods for demonstrating to a patient the effects of anti-reflective (AR) coatings on the patient's eyeglass lens prescription. The apparatus comprises a refractor, or a retrofit kit for a refractor, wherein at least the strong and weak sphere lenses are provided with a high index of refraction (IR) and are treated with an anti-reflective coating producing a high light transmission (LT) percentage and a low reflectance per surface (RPS) percentage. The apparatus further includes at least one filter which is removably placeable in viewing alignment with the viewing tube of the refractor. The filter is selected to have an IR, LT value and RPS value which, when disposed in alignment with the viewing tube in combination with any of the strong and/or weak sphere lenses produces a net LT value and net RPS value corresponding to the lenses to be used in the patients eyeglass lens without an AR coating treatment.

### CROSS REFERENCE TO RELATED APPLICATIONS

This application claims the benefit of, and priority to, U.S. Provisional Application No. 60/611,638 filed Sep. 21, 2004.

### BACKGROUND OF THE INVENTION

The chart of FIG. 7 identifies some of the various types of materials commonly used for eyeglass lenses. As identified in the chart, each of these materials has a known or definable index of refraction (“IR”), as well as a known or definable percentage of light transmittance (“LT”) and light reflectance per surface (“RPS”) for a given light wavelength. The values of the IR, LT and RPS identified in FIG. 7, are based on a light wavelength of 550 nm.

It should be understood that the higher a lenses LT percentage and the lower the lenses RPS percentage, the more light will pass through the lens to the eyes of the wearer and the less reflectance the wearer will experience. Thus, lenses with lower LT percentages and higher RPS percentages will cause the eyeglass wearer to receive less visible light and experience more internal and external light reflection through the lenses, which may result in mirror effects, ghost image effects and glare. Such effects are often pronounced with neon lights, when viewing computer or television screens or by vehicle headlights at night, causing discomfort and eye fatigue to the eyeglass wearer.

Thus, it should be appreciated that lenses with a high LT percentage and with a low RPS percentage are more desirable for eyeglasses. Of the lens materials identified in the chart of FIG. 7, glass and CR 39® have the highest LT percentage and the lowest RPS percentages. While these materials have good optical qualities for lenses, glass is a relatively heavy and brittle material. CR39® although lighter in weight and less brittle than glass, generally requires greater lens thicknesses to achieve a desired corrective lens prescription. In an effort to meet consumer demand for thinner and lighter weight lenses for eyeglasses, lens manufacturers began producing lenses from polycarbonates, hi-index plastics, hi-index glass and more recently super hi-index glass. Unfortunately, the LT percentages are generally lower and the RPS percentages generally higher with these higher-index materials than with glass and CR39®. However, as identified in the chart of FIG. 7, by coating the higher-index materials with a multi-layer anti-reflective (“AR”) coating, such as the Super ET® coating offered by Carl Zeiss, Inc. or other suitable AR coating treatment, the LT percentages and RPS percentages can be improved to meet or exceed the LT percentages and RPS percentages of glass and CR39® lenses.

Unfortunately, in the United States, only approximately twenty percent (20%) of all eyeglass lenses dispensed receive AR coatings despite the substantial benefits achieved with AR coatings. In other countries and regions, however, the majority of eyeglasses sold receive AR coatings. In Europe, for example, approximately seventy five percent (75%) of all eyeglass lenses dispensed receive AR coatings. In Japan, approximately ninety percent 90% of all eyeglass lenses dispensed receive AR coatings.

The reasons for such a low percentage of AR coated eyeglass sold in the United States, as opposed to other countries, may be due, in part, to the additional cost for AR coated lenses, as well as due to the lack of capital investment needed by the laboratories that process lens prescription orders to acquire the equipment and materials required to apply AR coatings to the lenses they process. However, it is submitted that the dominant reason many individuals elect not to purchase AR coated lenses for their eyeglasses is due to the fact that, heretofore, there has been no way for individuals to truly compare, at the point of sale, the difference in visual acuity between lenses with and without AR coatings. It is submitted that if most individuals are given the opportunity to truly experience the improvement in visual acuity achieved with AR coated lenses over non-AR coated lenses, most individuals will elect to receive AR coating treatment on their eyeglass lenses despite the increased cost.

Heretofore, optometrists, ophthalmologists and representatives of eyeglass retailers had to try to persuade their patients or customers to elect AR coatings on their lens by attesting to the benefits of AR coatings and relying on various types of point-of-sale displays including visual aids and demonstrative exhibits. Needless to say, most individuals who have not previously experienced the benefits of AR coating on their eyeglass lenses, are somewhat cynical when, at the point-of-sale, they are presented with a perceived “sales pitch” from the practitioner, and particularly from a sales representative of the retail store sales representative, touting the purported benefits of AR coating.

The cynicism of the customer is not dispelled by the currently available point-of-sale displays purporting to demonstrate the advantages of AR coated lenses. One type of point-of-sale display includes a photograph purporting to show the difference in visual acuity between side-by-side lenses, one having an AR coating and the other without. Another purports to demonstrate the cosmetic benefits of AR coated lenses, by a photograph showing a person wearing glasses with one lens, purporting to be the AR coated lens, appearing vary clear and transparent and the other lens, the non-AR coated lens, showing a glaring reflection so that the person's eye is not even visible. Yet another type of demonstrative exhibit that has been employed in the industry for promoting AR coatings at the point-of-sale is to provide a sample lens with one half of the lens treated with an AR coating. The problem with this type of demonstrative exhibit, however, is that it often raises the level of cynicism of the customer in that the lens half without the AR coating is usually scratched, smudged or is perceived by the customer as being an inferior quality lens, and thus does not accurately represent how the AR coating will truly benefit the customer with his/her particular lens prescription.

Accordingly, there is a need for apparatus and methods for demonstrating to individual prospective customers the benefits of applying AR coating to their eyeglass lenses which overcomes the cynicism and shortcomings associated with current apparatus and methods which purport to demonstrate the benefits of AR coatings on eyeglasses lenses.

### SUMMARY OF THE INVENTION

Apparatus and methods for demonstrating to a patient the effects of anti-reflective (AR) coatings on the patient's eyeglass lens prescription. The apparatus comprises a refractor, or a retrofit kit for a refractor, wherein at least the strong and weak sphere lenses are provided with a high index of refraction (IR) and are treated with an anti-reflective coating producing a high light transmission (LT) percentage and a low reflectance per surface (RPS) percentage. The apparatus further includes at least one filter which is removably placeable in viewing alignment with the viewing tube of the refractor. The at least one filter selected to have an IR, LT value and RPS value which, when disposed in alignment with the viewing tube in combination with any of the strong and/or weak sphere lenses produces a net LT value and net RPS value corresponding to the lenses to be used in the patients eyeglass lens without an AR coating treatment. The methods comprise the steps for demonstrating to a patient the effects of AR coatings on the patient's eyeglass lens prescription and methods for retrofitting refractors to enable such demonstrations.

### BRIEF DESCRIPTION OF THE DRAWINGS

FIG. 1 is a front view of a conventional refractor in which a preferred embodiment of the apparatus of the present invention is embodied, wherein the right eye battery is shown partially broken away to reveal internal gearing and support interconnecting the batteries.

FIG. 2 is a cross-sectional view of the right eye battery taken substantially along line 2-2 of FIG. 1, illustrating the right eye sphere lens assembly in elevation and the cylinder lens assembly and cross cylinder arrangement in cross-section.

FIG. 3 is a sectional view of the right eye battery as viewed along line 3-3 of FIG. 1, illustrating the right eye sphere assembly partially in section, the cylinder lens assembly in section and the cross cylinder arrangement partially in section.

FIG. 4 is an exploded perspective view of the weak sphere lens carrier disk of the refractor of FIG. 1.

FIG. 5 is an exploded perspective view of the strong sphere lens carrier disk of the refractor of FIG. 1.

FIG. 6 is an exploded perspective view of the auxiliary lens carrier disk of the refractor of FIG. 1, illustrating the filters for use in the apparatus and method of the present invention disposed for insertion into one of the blank apertures and for insertion into cells in place of the red lens and the +0.12D lens.

FIG. 7 is a chart identifying lens materials, and corresponding IR values, LT values, RPS values for the lens materials with and without AR-coating.

FIG. 8 illustrates an alternative embodiment for locating the filters in viewing alignment with the viewing tube.

### DETAILED DESCRIPTION OF THE INVENTION

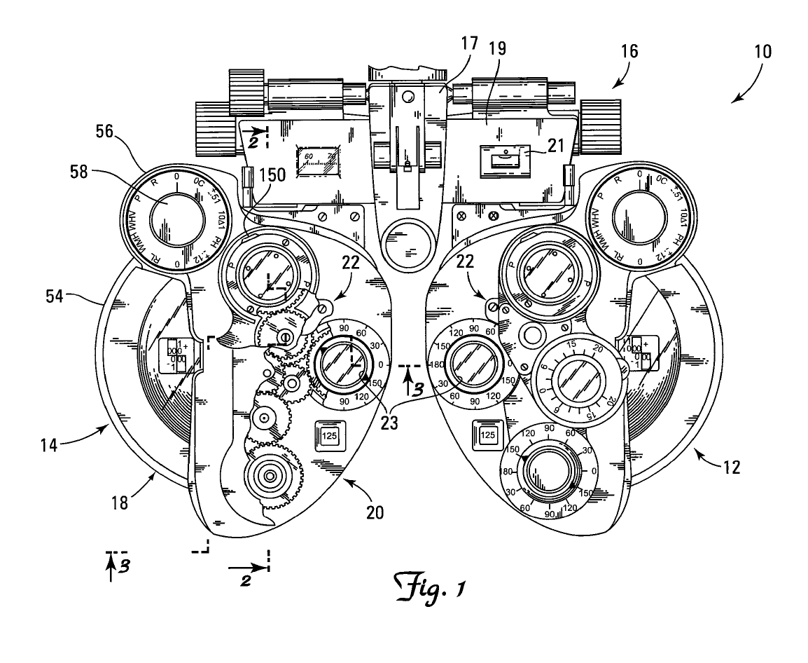

Drawing FIGS. 1 through 3 illustrate a refractor, designated generally by reference numeral 10, of the type disclosed in U.S. Pat. No. 3,498,699 issued to Wilkinson (hereinafter the “Wilkinson '699 patent”), which is hereby incorporated, in its entirety, by reference. A commercial embodiment of the refractor 10 disclosed in the Wilkinson '699 patent is presently manufactured and distributed by Reichert Ophthalmic Instruments under the trademark Phoroptor® (hereinafter referred to as the “Reichert Refractor”). While the apparatus and methods of the present invention for demonstrating the effects of AR lens coatings is particularly disclosed with respect to the Reichert Refractor, it should be understood that the apparatus and methods of the present invention are equally applicable to other types of refractors, whether now known or later developed. As a result, the apparatus and methods of the present invention should not be construed as being limited to or for use with any particular type of refractor except as otherwise specifically defined in the appended claims.

The refractor 10 includes a left eye battery 12 and a right eye battery 14. The two batteries 12, 14 are essentially mirror images of one another, and therefore only the components of a single battery are hereinafter discussed in detail. In FIG. 1, the refractor 10 is illustrated from the front or practitioner's side of the instrument. The patient's side of the instrument is hereinafter referred to as the rear side. The left and right batteries 12, 14 of the refractor 10 are retained side by side by a support 16. The support 16 permits desired manipulation of the batteries 12, 14 with respect to the patient's eyes and includes, generally, a yoke 17, a support bridge 19 and a level 21.

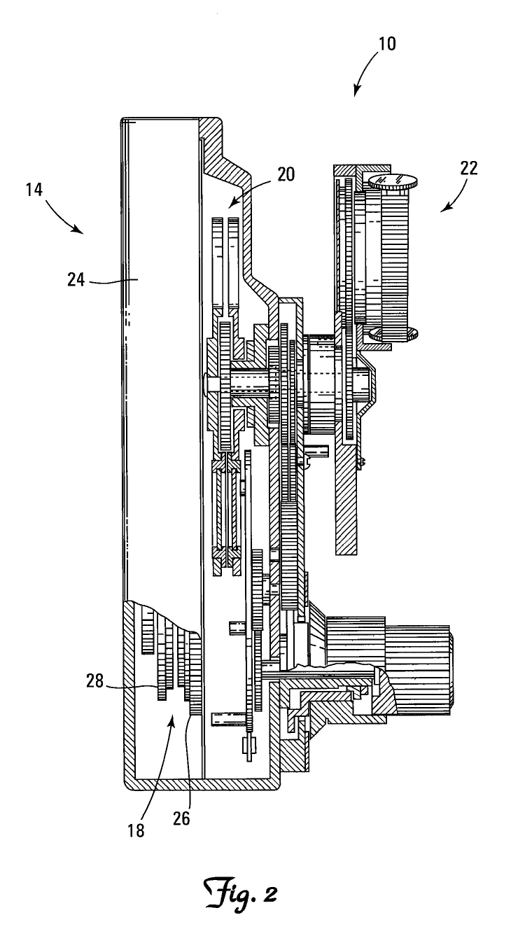

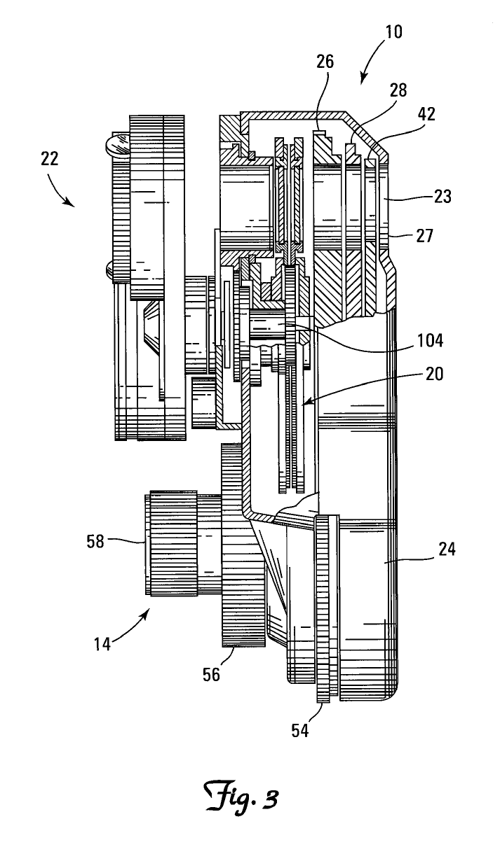

The major component parts of each battery 12, 14 are illustrated in FIGS. 2 and 3 and include a sphere lens assembly 18, a cylinder lens assembly 20 and a cross cylinder arrangement 22. Each of the batteries 12, 14 further includes a viewing tube 23. When in use, the patient's head is positioned to the rear of the instrument so that the patient's left and right eyes are positioned in substantial alignment with the left and right viewing tubes 23 of the respective left and right batteries 12, 14.

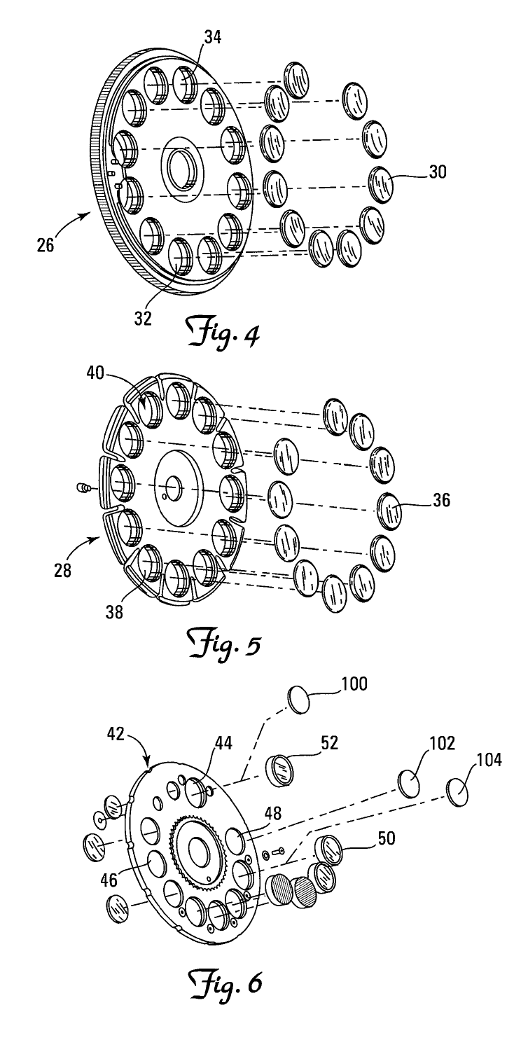

The sphere lens assembly 18 is best illustrated in FIGS. 2 and 3 and includes a sphere lens housing 24 in which a pair of lens carrier discs 26, 28 are coaxially rotatably mounted. The housing 24 includes a viewing aperture 27 which defines the rear end of the viewing tube 23. With reference to FIGS. 2 and 3, the forward-most lens carrier disc 26 carries a set of weak sphere lenses 30 and is therefore typically referred to in the industry as the “weak sphere disk.” As best illustrated in FIG. 4, which is an exploded perspective view of the weak sphere carrier disk 26, the disk includes a circular array of radially spaced cells 32, each successive cell 32 supporting an incrementally graded weak sphere lens 30. Typically one of the cells 32 is left vacant thereby defining a blank aperture 34. The rearward-most lens carrier disc 28 as illustrated in FIGS. 2 and 3 carries a set of strong sphere lenses 36 (FIG. 5) and is therefore typically referred to in the industry as the “strong sphere disk.” As best illustrated in FIG. 5, which is an exploded perspective view of the strong sphere carrier disk 28, the disk includes a circular array of radially spaced cells 38, each successive cell 38 supporting an incrementally graded strong sphere lens 36. Typically one of the cells 38 is left vacant thereby defining a blank aperture 40 (FIG. 4). The lenses 30, 36 of the lens carrier discs 26, 28 as well as the blank apertures 34, 40 are selectively and successively rotatable into viewing alignment with the viewing tube 23.

The sphere lens assembly 18 further includes an auxiliary lens carrier disc 42 disposed coaxial with the sphere lens discs 26, 28. The auxiliary disc 42 also includes a plurality of cells 44 as best illustrated in FIG. 6 which is an exploded perspective view of the auxiliary lens carrier disk 42. In the Reichert Refractor embodiment, two of the cells 44 are left vacant thereby defining blank apertures 46, 48. The remainder of the cells 44 typically support different types of auxiliary lenses including a red lens 50 and +0.12 diopter lens 52. As with the sphere lens discs 26, 28, the auxiliary lens disk 42 is also rotatable within the housing 24 such that each of the cells 44 can be selectively and successively rotated into viewing alignment with the viewing tube 23.

The selection of the desired cells 32, 38, 44 of the weak sphere disk 26, strong sphere disk 28 and auxiliary lens disk 42, respectively, for viewing alignment with the viewing tube is controlled, by rotation of the respective carrier disk. The weak sphere lens disc 26 is rotated by direct contact with its exposed knurled edge 54. The strong sphere lens carrier disk 28 is rotated by turning the strong sphere lens control knob 56. The auxiliary lens carrier disk is rotated by turning the auxiliary lens control knob 58. The internal structural components to effect the rotation of the disks 26, 28, 42 is more fully disclosed in U.S. Pat. No. 2,999,065, also incorporated herein by reference, in it entirety.

The construction and operation of the cylinder lens assembly 20 and cross cylinder arrangement 22 for the refractor 10 is fully set forth in the Wilkinson '699 patent, and in U.S. Pat. No. 2,968,213, also incorporated herein by reference, in it entirety. As such, no further discussion of the construction and operation of the refractor 10 in connection with the cylinder lens assembly 20 and cross cylinder arrangement 22 is provided herein; it being understood, however, that the construction and operation of the cylinder lens assembly 20 and cross cylinder arrangement 22 and all other features and functionalities of the refractor 10 as disclosed in Wilkinson '699 and the foregoing '065 and '213 patents, are to be considered disclosed herein as if expressly reprinted herein.

In the preferred embodiment of the apparatus of the present invention, the material used for the lenses 30, 36 of the weak and strong sphere disks 26, 28 and for any of the other lenses comprising the cylinder lens assembly 20 and cross cylinder arrangement 22, is preferably Super Hi-Index Glass with an IR value of 1.8 or greater. The lenses are treated with a AR coating, such as with the Super ET® multi-layer AR coating offered by Carl Zeiss, Inc. or some other suitable AR coating treatment. It should be appreciated, therefore, that with all of the lenses in the refractor 10 made of a material with one of the highest available indexes of refraction, and with each of the lenses treated with an AR coating, with the appropriate combination of lenses selected to correct the patient's vision deficiencies, the patient will be able to view a reference object through the viewing tube 23 under conditions approaching the greatest visual acuity possible with the patient's lens prescription.

In order to demonstrate to the patient the beneficial effects of the AR coating on the patient's eyeglass lenses, the perceived effects of the AR coating are removed from the lenses aligned in the viewing tube 23 by placing a filter into viewing alignment with the viewing tube 23. The filter acts to reduce the amount of light transmission through the viewing tube and increases the amount of light reflectance perceived by the patient so as to provide to the patient an accurate representation of the difference in visual acuity likely to be experienced if he/she elects to not receive AR coating on his/her prescribed lenses.

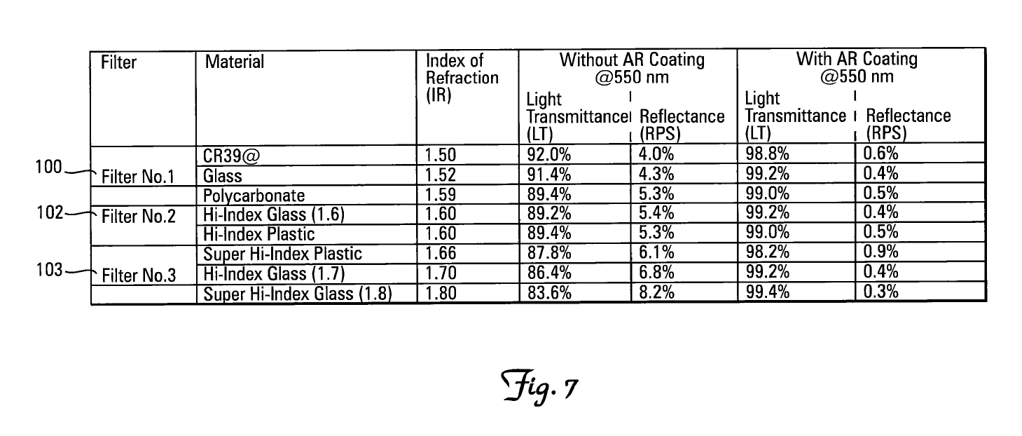

In the preferred embodiment, three filters 100, 102, 104 are preferably available for selection by the practitioner to provide the appropriate “corrections” to the IR value, LT value and RPS value of the AR coated lenses so as to accurately represent the patient's eyeglass lenses without an AR coating. Thus, as illustrated in FIG. 7, a first filter 100 preferably has an IR value, LT value and RPS value to produce a net IR value, net LT value and net RPS value in combination with the AR-coated lenses of the refractor 10 corresponding to the non-AR coated IR values, LT values and RPS values for glass and CR39® lens materials. The second filter 102 preferably has an IR value, LT value and RPS value to produce a net IR value, net LT value and net RPS value in combination with the AR-coated lenses of the refractor 10 corresponding to the non-AR coated IR values, LT values and RPS values for Polycarbonate, Hi-Index Glass (1.6) and Hi-Index Plastic lens materials. The third filter 104 preferably has an IR value, LT value and RPS value to produce a net IR value, net LT value and net RPS value in combination with the AR-coated lenses of the refractor 10 corresponding to the non-AR coated IR values, LT values and RPS values for Super Hi-Index Plastic and Hi-Index Glass (1.7).

It should be understood that only three filters as defined above are deemed necessary to provide the correction factors for each of the seven different materials presently used for eyeglass lenses. This is due to the fact that IR values, LT values and RPS values are so closely aligned when grouped as illustrated in FIG. 7 that any differences would likely not be perceptible to the patient.

In the preferred embodiment, the three filters 100, 102 and 104 are preferably disposed on the auxiliary lens carrier disk such that the filters can be selectively rotated into viewing alignment with the viewing tube by the practitioner rotating the auxiliary lens control knob 58 as previously described. With respect to the Reichert Refractor embodiment, one of the three filters 100, 102, 104 is preferably disposed in one of the blank apertures 46, 48, with the remaining two filters 102, 104 inserted into the cells 44 previously supporting the red lens and +0.12 diopter lens 52, which are rarely used by practitioners, and thus will not likely be missed by practitioners. Thus, it should be appreciated that by inserting the filters 100, 102, 104 into existing cells 44 in the auxiliary lens carrier disk 42, no cutting or other physical modification of the disk 42 is necessary, except to remove certain of the disk's existing lenses insertion of the filters into the available cells 44.

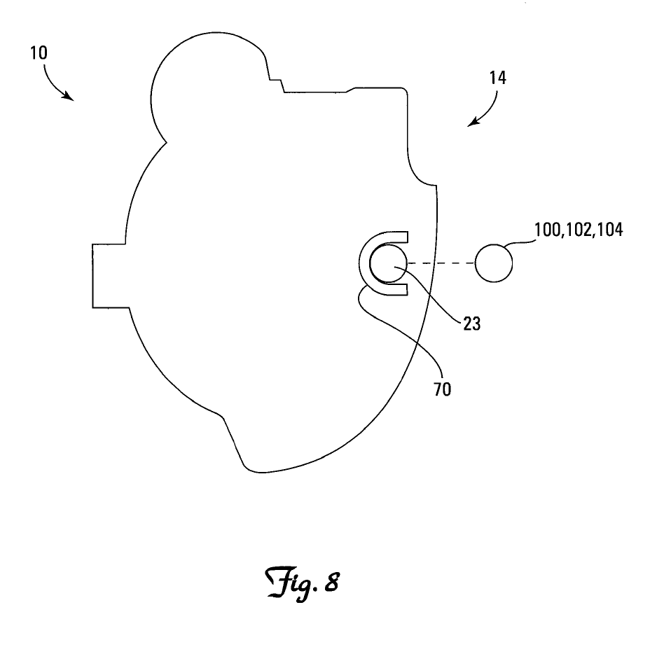

As an alternative embodiment, the filters 100, 102, 104 may be separate members adapted to be placed over the viewing tube, at the front or rear of the instrument, or both. As illustrated in FIG. 8, in such an alternative embodiment, a socket 70 may secured to the refractor, at the front or rear of the instrument or both, into which the filters 100, 102, 104 may be slidably inserted.

Using the foregoing preferred embodiment of the apparatus of the present, a preferred method of demonstrating effects of AR coatings on lenses to a patient is performed by the practitioner after the appropriate corrective lenses of the patient have been selected and with the selected AR-coated lenses still disposed in viewing alignment with the viewing tubes 23 of the right and left batteries 12, 14. With the patient looking through the viewing tubes, the practitioner selectively rotates the auxiliary lens carriers 42 so as to position in viewing alignment with each viewing tube 23, one of the filters 100, 102, 104 having the properties which will resulting in the net IR value, net LT value and net RPS value corresponding to the non-AR coated IR value, LT value and RPS value of the type of lenses to be used for the patient's prescription eyeglasses. With the patient continuing to look through the viewing tubes 23, the practitioner selectively rotates the auxiliary lens carriers 42 so as to remove the previously selected filters 100, 102, 104 from viewing alignment with the viewing tubes 23, whereupon the patient will again be able to perceived the reference object through the viewing tube 23 through the AR-coated lenses under the AR-coated IR value, the AR-coated LT value and AR-coated RPS value of the lenses. The foregoing steps can be repeated in succession as many times as necessary to enable the patient to compare the difference in visual acuity and other perceived effects with lens treated with an AR coating versus an accurate representation of the visual acuity and effects likely to be experienced with the same prescription lenses not treated with an AR coating.

As an alternative method of demonstrating the effects of AR-coated lenses, with respect to the alternative embodiment, instead of the practitioner rotating the auxiliary lens carrier with the filters disposed therein, the practitioner may simply insert the corresponding filter into the socket.

Although only certain exemplary embodiments of the apparatus and methods of present invention have been described in detail above, those skilled in the art will readily appreciate that many modifications are possible without materially departing from the novel teachings and advantages of this invention. Accordingly, all such modifications are intended to be included within the scope of this invention as defined in the following claims.

1. A refractor, comprising:
a left eye battery and a right eye battery, each eye battery including:
a housing;
a viewing tube through said housing;
first and second lens carrier disks, said first lens carrier disk supporting a set of weak sphere lenses, said second lens carrier disk supporting a set of strong sphere lenses, each of said lenses supported by said first and second lens carriers made of a lens material having an anti-reflective (AR) coating, said AR-coated lens material producing a AR-coated light transmittance (LT) value and a AR-coated reflectance (RPS) value, said first and second lens carrier disks rotatable about an axis wherein said AR-coated lenses are each selectively and successively rotatable into coaxial viewing alignment with said viewing tube;
at least one filter removably placeable in viewing alignment with said view tube, said filter having a filter IR value, filter LT value and filter RPS value selected so as to produce, when placed in viewing alignment with said viewing tube having any combination of said lenses of said first and second lens carriers in viewing alignment therewith, a net RT value and a net RPS value substantially corresponding to a known LT value and a known RPS value for said lens material without an AR coating.

2. The refractor of claim 1 wherein each said eye battery further includes an auxiliary lens disc carrier and wherein said at least one filter is disposed in a cell of said auxiliary lens disc carrier, said auxiliary lens disk carrier rotatable about an axis wherein said first filter is rotatable into coaxial viewing alignment with said viewing tube.

3. The refractor of claim 2 wherein said AR coated lenses of said first and second lens carrier disks have, with light wavelengths of about 550 nm, a LT value in a range of approximately 98% to approximately 99% and an RPS value in a range of approximately 0.3% to approximately 0.9%.

4. The refractor of claim 3 wherein said at least one first filter of said auxiliary lens disk has, with light wavelengths of about 550 nm, a filter LT value of approximately 89% and a filter RPS value of approximately 5%.

5. The refractor of claim 3 wherein said at least one first filter of said auxiliary lens disk has, with light wavelengths of about 550 nm, a filter LT value of approximately 87% and a filter RPS value of approximately 6%.

6. The refractor of claim 3 wherein said at least one first filter of said auxiliary lens disk has, with light wavelengths of about 550 nm, a filter LT value of approximately 92% and filter RPS value of approximately 4%.

7. The refractor of claim 4 wherein said auxiliary lens disk carrier further includes a second filter and a third filter, wherein:
said second filter has, with light wavelengths of about 550 nm, a filter LT value of approximately 87% and a filter RPS value of approximately 6%; and
said third filter has, with light wavelengths of about 550 nm, a filter LT value of approximately 92% and filter RPS value of approximately 4%.

8. A retrofit kit for refractors of a type having a left eye battery and a right eye battery, each battery including a housing, a viewing tube through the housing, a weak sphere lens carrier disk, a strong sphere lens carrier disk said weak, strong and auxiliary lens carrier disks, having cells selectively and successively rotatable into coaxial viewing alignment with said viewing tube, the retrofit kit comprising:
a first lens carrier disk substantially identical to the weak sphere lens carrier disk of the refractors, said first lens carrier disk having cells supporting a plurality of weak sphere lenses and an aperture;
a second lens carrier disk substantially identical to the strong sphere lens carrier disk of the refractors, said second lens carrier disk having cells supporting a plurality of strong sphere lenses and an aperture, wherein each of said weak sphere lenses and said strong sphere lenses are made of a lens material, said lens material having an AR coating, said AR-coated lens material producing a AR-coated LT value and a AR-coated RPS value;
a first filter removably placeable into viewing alignment with said viewing tube, said first filter made of filter material having a filter IR value, filter LT value and filter RPS value selected so as to produce through said viewing tube, when disposed in coaxial viewing alignment therewith and in combination with any said cells of said first and second lens carriers, a net RT value and a net RPS value substantially corresponding to a known LT value and known RPS value for said lens material absent said AR coating.

9. The retrofit kit of claim 8 wherein the refractors further include an auxiliary lens carrier disk, and wherein the retrofit kit further comprises a third lens carrier disk substantially identical to the auxiliary lens carrier disk of the refractors, said third lens carrier disk having at least one cell supporting said first filter.

10. The retrofit kit of claim 9 wherein said AR coated lens material produces, with light wavelengths of about 550 nm, an AR-coated LT value in a range of approximately 98% to approximately 99% and a AR-coated RPS value in a range of approximately 0.3% to approximately 0.9%.

11. The retrofit kit of claim 10 wherein said first filter of said third lens carrier disk has, with light wavelengths of about 550 nm, a filter LT value of approximately 89% and a filter RPS value of approximately 5%.

12. The retrofit kit of claim 10 wherein said first filter of said third lens carrier disk has, with light wavelengths of about 550 nm, a filter LT value of approximately 87% and a filter RPS value of approximately 6%.

13. The retrofit kit of claim 10 wherein said first filter of said third lens carrier disk has, with light wavelengths of about 550 nm, a filter LT value of approximately 92% and filter RPS value of approximately 4%.

14. The retrofit kit of claim 11 wherein said third lens carrier disk further includes other cells supporting at least a second filter and a third filter, wherein:
said second filter has, with light wavelengths of about 550 nm, a filter LT value of approximately 87% and a filter RPS value of approximately 6%; and
said third filter has, with light wavelengths of about 550 nm, a filter LT value of approximately 92% and filter RPS value of approximately 4%.

15. A method of demonstrating effects of AR coatings on lenses to a patient, said method comprising the steps of:
(a) providing a refractor, of a type having a left eye battery and a right eye battery, each battery including:
(i) a housing;
(ii) a viewing tube through said housing;
(iii) a weak sphere lens carrier disk rotatably disposed in said housing;
(iv) a strong sphere lens carrier disk rotatably disposed in said housing; and
wherein said weak and strong lens carrier disks each include cells selectively and successively rotatable into coaxial viewing alignment with said viewing tube, certain of said cells of said weak and strong lens carrier disks having lenses disposed therein, said lenses made of a lens material, said lens material having an AR coating, said AR-coated lens material producing a AR-coated LT value and a AR-coated RPS value;
(b) providing at least one filter, said at least one filter made of filter material having a filter IR value, filter LT value and filter RPS value, selected so as to produce through said viewing tube, when disposed in coaxial viewing alignment with any combination of said lenses of said weak and strong lens carrier disks, a net RT value and a net RPS value substantially corresponding to a known LT value and known RPS value for said lens material absent said AR coating;
(c) positioning the refractor with respect to the patient such that said viewing tubes of said left and right batteries are in substantial alignment with said patient's line of sight;
(d) selectively rotating at least one of said weak and strong lens carrier disks to position at least one AR-coated lens in viewing alignment with said viewing tube, whereby an object in said patient's line of site is perceived by said patient through said AR-coated lens under said AR-coated LT value and a said AR-coated RPS value;
(e) placing said at least one filter in viewing alignment with said viewing tube, whereby the object in said patient's line of site is perceived by said patient through said at least one filter thereby producing said net RT value and said net RPS value to the patient;
(f) removing said at least one filter from viewing alignment with said viewing tube, whereby the object in said patient's line of site is again perceived by said patient under said AR-coated LT value and a said AR-coated RPS value;
(g) repeating steps (e) and (f) in succession to enable the patient to compare between the object perceived through said lens material having said AR-coating versus through said lens material absent said AR coating.

16. The method of claim 15 wherein each battery of the refractor further includes an auxiliary lens carrier disk rotatably disposed in said housing, said auxiliary lens carrier disk having a plurality of cells at least one of said cells supporting said at least one filter.

17. The method of claim 16 wherein said step (e) includes rotating said auxiliary lens carrier disk to position said at least one filter supported thereby in viewing alignment with said viewing tube and said step (f) includes rotating said auxiliary lens carrier disk to remove said at least one filter from viewing alignment with said viewing tube.

18. The method of claim 15 wherein said AR coated lenses of said weak sphere and said strong sphere lens carrier disks have, with light wavelengths of about 550 nm, a LT value in a range of approximately 98% to approximately 99% and an RPS value in a range of approximately 0.3% to approximately 0.9%.

19. The method of claim 17 wherein said AR coated lenses of said weak sphere and said strong sphere lens carrier disks have, with light wavelengths of about 550 nm, a LT value in a range of approximately 98% to approximately 99% and an RPS value in a range of approximately 0.3% to approximately 0.9%.

20. The method of claim 19 wherein said at least one filter has, with light wavelengths of about 550 nm, a filter LT value of approximately 89% and a filter RPS value of approximately 5%.

21. The method of claim 19 wherein said at least one filter has, with light wavelengths of about 550 nm, a filter LT value of approximately 87% and a filter RPS value of approximately 6%.

22. The method of claim 19 wherein said at least one filter has, with light wavelengths of about 550 nm, a filter LT value of approximately 92% and filter RPS value of approximately 4%.

23. The method of claim 20 wherein said auxiliary lens carrier disk includes at least two other cells in which is disposed a second filter and a third filter, wherein:
said second filter has, with light wavelengths of about 550 nm, a filter LT value of approximately 87% and a filter RPS value of approximately 6%; and
said third filter has, with light wavelengths of about 550 nm, a filter LT value of approximately 92% and filter RPS value of approximately 4%.

24. A method for retrofitting a refractor to demonstration to a patient effects of AR coatings on lenses, wherein the refractor being retrofitted is of a type having a left eye battery and a right eye battery, each battery including a housing, a viewing tube through the housing, a weak sphere lens carrier disk and a strong sphere lens carrier disk, each having cells selectively and successively rotatable into coaxial viewing alignment with said viewing tube, the method for retrofitting comprising:
(a) removing from the refractor being retrofitted, the weak sphere, the strong sphere and the auxiliary lens carrier disks;
(b) providing a first lens carrier disk substantially identical to the weak sphere lens carrier disk of the refractor being retrofitted, said first lens carrier disk having cells supporting a plurality of weak sphere lenses and an aperture;
(c) providing a second lens carrier disk substantially identical to the strong sphere lens carrier disk of the refractor being retrofitted, said second lens carrier disk having cells supporting a plurality of strong sphere lenses and an aperture, wherein each of said weak sphere lenses and said strong sphere lenses are made of a lens material, said lens material having an AR coating, said AR-coated lens material producing a AR-coated LT value and a AR-coated RPS value;
(d) providing at least one filter having a filter IR value, filter LT value and filter RPS value selected so as to produce when disposed in coaxial viewing alignment with any combination of said cells said first and second lens carriers, a net RT value and a net RPS value substantially corresponding to a known LT value and known RPS value for said lens material absent said AR coating;
(e) replacing the removed weak sphere and strong sphere with the corresponding one of said first and second lens carrier disks in the refractor being retrofitted.

25. The method of claim 24 wherein said AR-coated lens material produces, with light wavelengths of about 550 nm, an AR-coated LT value in a range of approximately 98% to approximately 99% and a AR-coated RPS value in a range of approximately 0.3% to approximately 0.9%.

26. The method of claim 24 wherein each battery of the refractor being retrofitted further includes an auxiliary lens carrier disk and wherein the method further comprises the steps of:
(f) providing a third lens carrier disk substantially identical to the auxiliary lens carrier disk of the refractor being retrofitted, said third lens carrier disk having one cell supporting said at least one filter;
(g) replacing the auxiliary lens carrier disk with said third lens carrier disk.

27. The method of claim 26 wherein said AR-coated lens material produces, with light wavelengths of about 550 nm, an AR-coated LT value in a range of approximately 98% to approximately 99% and a AR-coated RPS value in a range of approximately 0.3% to approximately 0.9%.

28. The method of claim 27 wherein said at least one filter of said third lens carrier disk has, with light wavelengths of about 550 nm, a filter LT value of approximately 89% and a filter RPS value of approximately 5%.

29. The method of claim 27 wherein said at least one filter of said third lens carrier disk has, with light wavelengths of about 550 nm, a filter LT value of approximately 87% and a filter RPS value of approximately 6%.

30. The method of claim 27 wherein said at least one filter of said third lens carrier disk has, with light wavelengths of about 550 nm, a filter LT value of approximately 92% and filter RPS value of approximately 4%.

31. The method of claim 28 wherein said third lens carrier disk further includes a second cell supporting a second filter and a third cell supporting a third filter, wherein:
said second filter has, with light wavelengths of about 550 nm, a filter LT value of approximately 87% and a filter RPS value of approximately 6%; and
said third filter has, with light wavelengths of about 550 nm, a filter LT value of approximately 92% and filter RPS value of approximately 4%.
